(19) United States
(12) Patent Application Publication
(10) Pub. No.: US 2025/0032034 A1
(43) Pub. Date: Jan. 30, 2025

(54) METHOD AND APPARATUS FOR DETECTING ATRIAL FIBRILLATION BY USING DEEP LEARNING

(71) Applicant: SEERSTECHNOLOGY CO.,LTD., Seongnam-si, Gyeonggi-do (KR)

(72) Inventors: Youngshin LEE, Yongin-si, Gyeonggi-do (KR); Hoohyun KIM, Guri-si, Gyeonggi-do (KR); Heeseok SONG, Hwaseong-si, Gyeonggi-do (KR); Kunwook CHO, Seongnam-si, Gyeonggi-do (KR)

(73) Assignee: SEERSTECHNOLOGY CO.,LTD., Seongnam-si, Gyeonggi-do (KR)

(21) Appl. No.: 18/715,717

(22) PCT Filed: Dec. 17, 2021
(86) PCT No.: PCT/KR2021/019295

§ 371 Date: Jul. 3, 2024

(30) Foreign Application Priority Data
Dec. 2, 2021 (KR) 10-2021-0170500

(51) Int. Cl. A61B 5/361; A61B 5/00; A61B 5/352; A61B 5/353; A61B 5/355; A61B 5/364

(52) U.S. Cl. CPC A61B 5/361; A61B 5/352; A61B 5/353; A61B 5/355; A61B 5/364; A61B 5/7267; A61B 5/7282

(57) ABSTRACT

Disclosed is an atrial fibrillation discriminating apparatus using deep learning being capable of performing learning whether atrial fibrillation, which is a type of arrhythmia, occurs on the basis of deep learning, by applying a segmentation scheme to an ECG wave to quickly classify fibrillation waves on the basis of an ROI (Region of Interest) section in an ECG wave, thereby detecting the atrial fibrillation.

### TECHNICAL FIELD

The present invention relates to an apparatus for detecting an F wave of an electrocardiogram (ECG) and atrial fibrillation using a deep learning-based segmentation algorithm.

### BACKGROUND ART

The information disclosed in this background section is only for enhancement of understanding of the background of the invention, and does not define the prior art.

An electrocardiogram (ECG) reading system that allows a medical staff to analyze an ECG has been developed. A conventional ECG reading system detects R, P, and T peaks of an ECG wave, and detects and classifies arrhythmia in a rule-based manner.

Such an ECG reading system receives patient's entire ECG signal data, analyzes the data, and outputs its result. A deep learning technology has recently been widely studied for an ECG reading algorithm due to its high accuracy.

Only a qualified medical staff can determine arrhythmia using an ECG, but there is a shortage of manpower compared with demand. In reading the ECG, it takes a lot of time since an ECG signal must be read from various perspectives, such as calculation of time differences between shapes and sections of P, QRS, and T waves, and analysis of ECG rhythms. A medical staff must observe a patient's ECG in real time to keep an eye on patient's conditions, but continuous monitoring is difficult due to a lack of manpower. Since the ECG analysis is directly related to the patient's life, the analysis must be operated accurately and quickly in an emergency.

The conventional ECG analysis may use end and start points of P, QRS, and T wave sections, but merely searches for peaks, which results in low usability. In detection and classification of arrhythmia, a typical rule-based algorithm design is less accurate due to diversity of waves, and thus, it is necessary to design a new rule-based algorithm in consideration of arrhythmia.

Particularly, in a case where real-time reading is necessary, such as bedside patient monitoring, it is difficult to use such a conventional ECG reading system. Deep learning algorithms for ECG analysis have different speeds depending on implementation models, and may not easily perform real-time operation every moment. In visualizing the ECG waves, one-dimensional data is output, which lowers readability during real-time reading.

Since atrial fibrillation data has very diverse waves depending on individuals, there is a limitation in detecting the atrial fibrillation based on the waves. Therefore, in a case where atrial fibrillation occurs, there is a need for technique capable of determining an occurrence section of atrial fibrillation by detecting accompanying fibrillation waves.

### DISCLOSURE

### Technical Problem

Therefore, the present invention has been made in view of the above problems, and it is an object of the present invention to provide an atrial fibrillation discriminating apparatus using deep learning, capable of performing learning whether atrial fibrillation, which is a type of arrhythmia, occurs on the basis of deep learning, by applying a segmentation scheme to an ECG wave to quickly classify fibrillation waves on the basis of an ROI (Region of Interest) section in an ECG wave, thereby detecting the atrial fibrillation.

### Technical Solution

In accordance with an aspect of the present invention, there is provided by an atrial fibrillation discriminating apparatus including an ECG wave acquisition unit that acquires an ECG wave for each of a plurality of a person, a classification unit that applies segmentation to the ECG wave to check respective sections of the ECG wave, and labels classification values for the sections, a fibrillation selection unit that selects only a fibrillation wave labeled as tremors on the basis of the classification values for the respective sections, and an atrial fibrillation determination unit that determines atrial fibrillation in a case where the fibrillation wave of a preset threshold or greater is included.

### Advantageous Effects

According to the present embodiment as described above, it is possible to perform learning whether atrial fibrillation, which is a type of arrhythmia, occurs on the basis of deep learning, by applying a segmentation scheme to an ECG wave to quickly classify a fibrillation wave on the basis of an ROI (Region of Interest) section in the ECG wave, thereby detecting the atrial fibrillation.

Further, it is possible to reduce a reading burden on a medical staff and a reading time, and to perform continuous management through accurate detection of atrial fibrillation, and thus, it is possible to make an appropriate response in the event of an emergency.

In addition, it is possible to detect various atrial fibrillation cases, and in a case where a false detection occurs, it is possible to update an atrial fibrillation detection line by cutting only a false detection occurrence section and inputting the section as learning data.

### DESCRIPTION OF DRAWINGS

FIG. 1 is a diagram showing application of semantic segmentation to bio-signal data processing in a bio-signal processing field according to an embodiment of the present invention.

FIG. 2 is a diagram showing a point-by-point output of semantic segmentation according to an embodiment of the present invention.

FIG. 3 is a diagram showing electrocardiogram (ECG) semantic segmentation according to an embodiment of the present invention.

FIG. 4 is a diagram showing P, Q, R, S, and T waves (P wave, QRS complex, and T wave) and ECG features according to an embodiment of the present invention.

FIG. 5 is a diagram schematically showing an atrial fibrillation discriminating apparatus according to an embodiment of the present invention.

FIG. 6 is a diagram showing 2D image segmentation and 1D bio-signal segmentation according to an embodiment of the present invention.

FIG. 7 is a diagram showing an F wave in an atrial fibrillation section according to an embodiment of the present invention.

FIG. 8 is a diagram showing point-by-point annotation according to an embodiment of the present invention.

FIG. 9 is a diagram showing a structure of an encoder-decoder segmentation algorithm according to an embodiment of the present invention.

FIG. 10 is a diagram showing a process of outputting a final output value for each point according to an embodiment of the present invention.

FIG. 11 is a diagram showing a start point and an end point of an F wave according to an embodiment of the present invention.

FIG. 12 is a diagram showing point-by-point annotation according to an embodiment of the present invention.

### DESCRIPTION OF REFERENCE NUMERALS

500: ATRIAL FIBRILLATION DISCRIMINATING APPARATUS
510: ECG WAVE ACQUISITION UNIT
520: CLASSIFICATION UNIT
530: FIBRILLATION SELECTION UNIT
540: ATRIAL FIBRILLATION DETERMINATION UNIT
550: LEARNING UNIT

### BEST MODE

Hereinafter, the present embodiments will be described in detail with reference to the accompanying drawings.

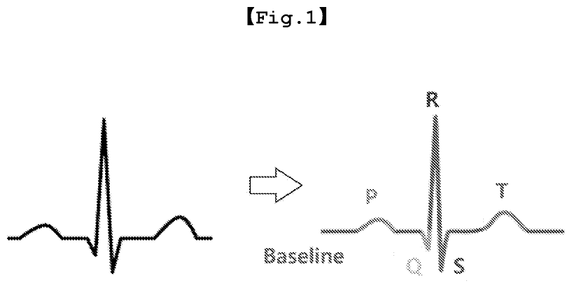

FIG. 1 is a diagram showing application of semantic segmentation to bio-signal data processing in a bio-signal processing field according to an embodiment of the present invention.

An atrial fibrillation discriminating apparatus 500 according to the present embodiment may be applied to one-dimensional (1D) bio-signal data processing in the field of bio-signal processing. The atrial fibrillation discriminating apparatus 500 classifies a fibrillation wave included in an electrocardiogram (ECG) wave in the unit of waves, using semantic segmentation.

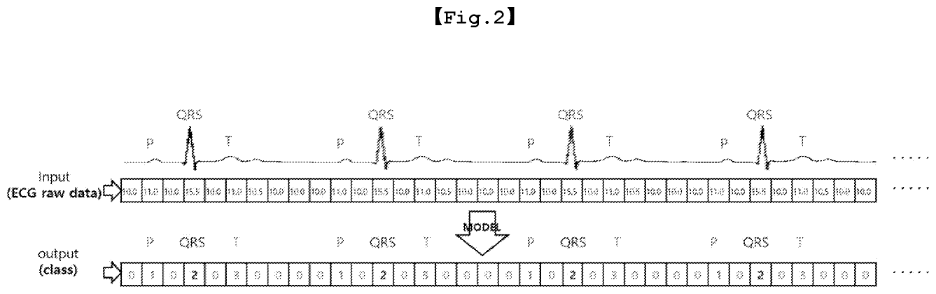

FIG. 2 is a diagram showing a point-by-point output of semantic segmentation according to an embodiment of the present invention.

The atrial fibrillation discriminating apparatus 500 according to the present embodiment outputs values obtained by classifying an ECG wave (raw data) in a point-by-point manner using a 1D semantic segmentation algorithm.

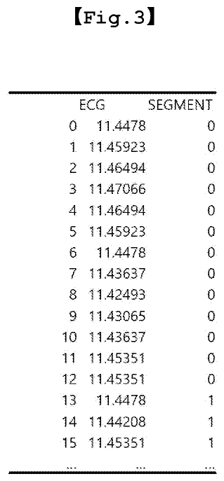

FIG. 3 is a diagram showing electrocardiogram (ECG) semantic segmentation according to an embodiment of the present invention.

In classifying the input ECG wave (raw data) in the point-by-point manner using the 1D semantic segmentation algorithm, the atrial fibrillation discriminating apparatus 500 sets the value as 1 in a case where the wave corresponds to atrial fibrillation, and sets the value as 0 in a case where the wave is a normal wave other than the atrial fibrillation.

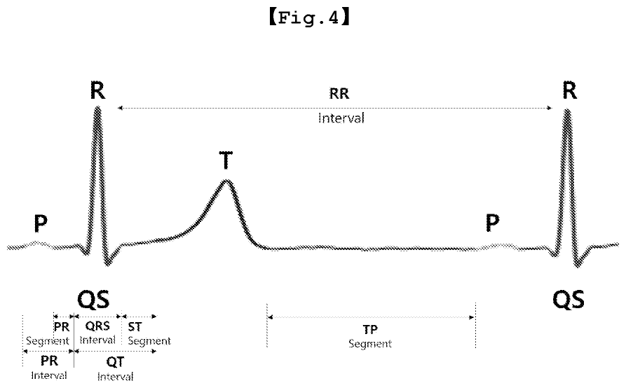

FIG. 4 is a diagram showing P, Q, R, S, and T waves (P wave, QRS complex, and T wave) and ECG features according to an embodiment of the present invention.

The ECG wave appears as a series of beats. The beats may be broadly classified into a normal beat (N), a supraventricular beat (S), and a ventricular beat (V). One beat of the ECG wave basically includes the P wave, the QRS wave, and the T wave.

The atrial fibrillation discriminating apparatus 500 detects the P wave, the Q wave, the R wave, the S wave, and the T wave included in the ECG wave, and classifies the beats into the normal beat (N), the supraventricular beat (S), and the ventricular beat (V). The atrial fibrillation discriminating apparatus 500 outputs various features by performing localization on the P wave, the Q wave, the R wave, the S wave, and the T wave.

The atrial fibrillation discriminating apparatus 500 distinguishes a PR interval, a QRS interval, a QT interval, an ST segment, and an RR interval in the input ECG wave.

The atrial fibrillation discriminating apparatus 500 may classify the beats on the basis of feature information of the P wave, the Q wave, the R wave, the S wave, and the T wave, on the basis of the PR interval, the QRS interval, the QT interval, the ST segment, and the RR interval.

As described above, the atrial fibrillation discriminating apparatus 500 classifies heart beats into the normal beat (N), the supraventricular beat (S), and the ventricular beat (V). In reading the ECG wave, the atrial fibrillation discriminating apparatus 500 may detect an abnormal state on the basis of information on the localization and classification.

The atrial fibrillation discriminating apparatus 500 detects an abnormal state (arrhythmia, abnormal rhythms (S, V), ST, QTc, etc.) using values obtained by performing localization on the ECG wave.

The atrial fibrillation discriminating apparatus 500 performs classification on the ECG wave, and classifies heart beats into the normal beat (N), the supraventricular beat (S), and the ventricular beat (V) to thereby detect arrhythmia.

The atrial fibrillation discriminating apparatus 500 applies a segmentation technique to the ECG wave to check respective sections of the P wave, the Q wave, the R wave, the S wave, the T wave, and the atrial fibrillation wave included in the ECG wave. The atrial fibrillation discriminating apparatus 500 quickly classifies the heart beats into the normal beat (N), the supraventricular beat (S), the ventricular beat (V), and the atrial fibrillation on the basis of the respective sections.

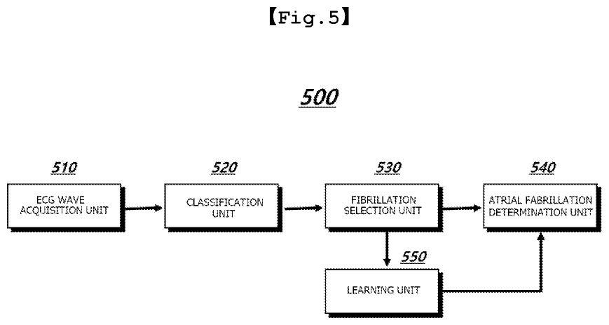

FIG. 5 is a diagram schematically showing an atrial fibrillation discriminating apparatus according to an embodiment of the present invention.

A medical staff may adjust a threshold of an occurrence rate to check a probable candidate group and a general candidate group as atrial fibrillation, using the atrial fibrillation discriminating apparatus 500.

In a case where the medical staff sets the threshold high (for example, 90%), since a very probable candidate group as atrial fibrillation (=candidate section) is first shown in a situation where relatively short reading is necessary, the medical staff may quickly monitor and handle a patient's condition.

In a case where the medical staff sets the threshold low (for example, 40%), since all probable candidate sections as atrial fibrillation are shown to the medical staff in a situation where detailed reading is necessary as in general health examinations, it is possible to perform precise reading.

The atrial fibrillation discriminating apparatus 500 according to the present embodiment includes an ECG wave acquisition unit 510, a classification unit 520, a fibrillation selection unit 530, an atrial fibrillation determination unit 540, and a learning unit 550. The respective components included in the atrial fibrillation discriminating apparatus 500 are not necessarily limiting.

The respective components included in the atrial fibrillation discriminating apparatus 500 may be connected to a communication path that connects software modules or hardware modules within the apparatus to be operated in combination with each other. These components perform communication using one or more communication buses or signal lines.

Each component of the atrial fibrillation discriminating apparatus 500 shown in FIG. 5 refers to a unit that processes at least one function or operation, and may be implemented as a software module, a hardware module, or a combination of software and hardware.

The ECG wave acquisition unit 510 acquires data, and converts the data into one-dimensional data.

The ECG wave acquisition unit 510 acquires ECG waves for a plurality of persons. The ECG wave acquisition unit 510 acquires 256 samples of ECG waves per second for each of the plurality of persons. The ECG wave acquisition unit 510 converts the ECG waves of the plurality of persons into one-dimensional data.

The classification unit 520 outputs a model classification result value (0 or 1) for each point.

The classification unit 520 applies segmentation to the ECG wave and checks respective sections of the ECG wave. The classification unit 520 labels classification values for the respective sections.

The classification unit 520 checks the respective sections of a P wave, a Q wave, an R wave, an S wave, a T wave, and an ROI (Region of Interest) included in the ECG wave.

In a case where the fibrillation wave of a preset threshold (for example, 50%) or greater is detected in the ROI section of the ECG wave and is determined as the atrial fibrillation, the classification unit 520 labels the ROI section as 1. In a case where the fibrillation wave is detected below the preset threshold (for example, 50%) in the ROI section of the ECG wave and is determined as non-atrial fibrillation, the classification unit 520 labels the ROI section as 0. The classification unit 520 labels the section having the value of 1 as the fibrillation wave.

After the labeling is completed, the classification unit 520 moves the ROI section to overlap on the ECG wave to thereby confirm exact start and end points of the atrial fibrillation. After the labeling is completed, the classification unit 520 detects the fibrillation wave while moving the ROI section in a preset time unit (for example, about 1 second).

The classification unit 520 determines a point at which the atrial fibrillation wave is first detected in the ROI section as the start point. The classification unit 520 determines a point at which the atrial fibrillation wave is not detected as the end point of the atrial fibrillation while moving the ROI section in the preset time unit.

The classification unit 520 detects the atrial fibrillation wave according to an irregularity of an RR interval between the R wave and the next R wave and a morphological shape representing baseline fluctuation, in beat cycles of the ECG wave.

The fibrillation selection unit 530 selects the fibrillation wave (value 1). The fibrillation selection unit 530 selects only a fibrillation wave labeled as tremors on the basis of the classification values for the respective sections.

The atrial fibrillation determination unit 540 determines whether atrial fibrillation occurs (Yes or No) in a 10-second section, and outputs the result. In a case where the fibrillation wave of a preset threshold (for example, 50%) or greater is detected, the atrial fibrillation determination unit 540 determines that the atrial fibrillation has occurred. The atrial fibrillation determination unit 540 determines whether the atrial fibrillation has occurred by checking the proportion of the wave determined as the atrial fibrillation and labeled as 1 in the ROI section in the whole ECG wave.

The learning unit 550 performs learning using only the atrial fibrillation wave as input to generate an atrial fibrillation learning result.

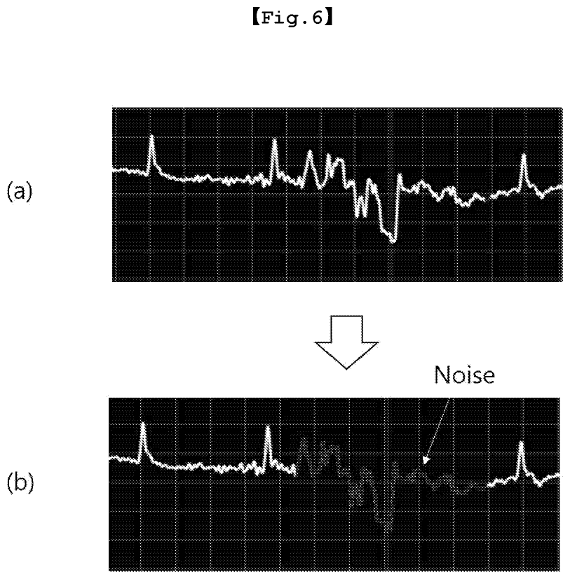

FIG. 6 is a diagram showing 2D image segmentation and 1D bio-signal segmentation according to an embodiment of the present invention.

The atrial fibrillation discriminating apparatus 500 applies the segmentation algorithm to the ECG wave (1D data) to segment the ECG wave into semantic units.

The atrial fibrillation discriminating apparatus 500 detects and classifies the ECG wave using the segmentation algorithm. The atrial fibrillation discriminating apparatus 500 accurately distinguishes respective sections in the ECG wave using the segmentation algorithm.

The atrial fibrillation discriminating apparatus 500 may classify respective sections not only in the ECG but also in various bio-signals using the segmentation algorithm.

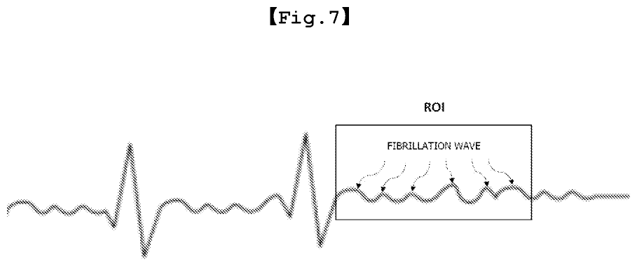

FIG. 7 is a diagram showing an F wave in an atrial fibrillation section according to an embodiment of the present invention.

In most cases, a typical atrial fibrillation detection algorithm detects atrial fibrillation on the basis of the shape of a wave, but the atrial fibrillation discriminating apparatus 500 according to the present embodiment confirms atrial fibrillation in a case where a baseline is accompanied by an irregular fibrillation wave, and indirectly determines a section where a fibrillation wave of a predetermined ratio or greater is detected as an atrial fibrillation section.

In general, in determining arrhythmia, a section where there is an irregularity in the RR interval in the ECG wave is classified as arrhythmia, or in a case where the P wave occurs consecutively, it is determined that the heart beats irregularly. In other words, arrhythmia is determined on the basis of the P wave in the ECG wave.

The atrial fibrillation discriminating apparatus 500 performs labeling for the ECG wave using the segmentation algorithm. Unlike the general technique of checking the irregularity of the RR interval in the ECG wave to determine arrhythmia, the atrial fibrillation discriminating apparatus 500 according to the present embodiment determines the irregularity of the RR interval and the morphological shape of the baseline fluctuation using the segmentation algorithm.

The atrial fibrillation discriminating apparatus 500 performs classification of atrial fibrillation by performing labeling for the ECG wave using the segmentation algorithm.

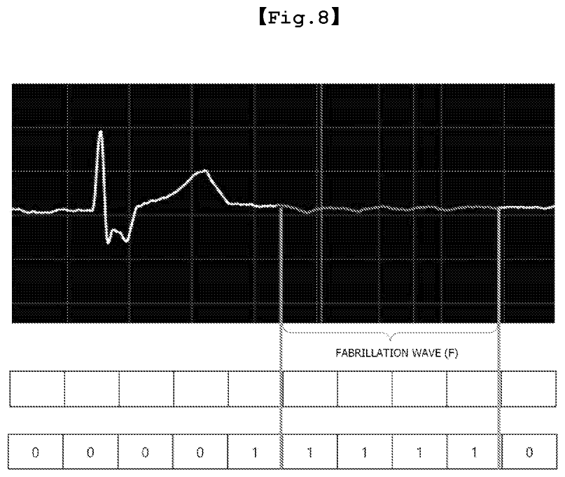

FIG. 8 is a diagram showing point-by-point annotation according to an embodiment of the present invention.

The atrial fibrillation discriminating apparatus 500 prepares learning data for learning a deep learning model.

The atrial fibrillation discriminating apparatus 500 performs annotation for a section determined as a fibrillation wave and sections other than the fibrillation wave in raw data as different classes in a point-by-point manner.

The atrial fibrillation discriminating apparatus 500 annotates a point where the fibrillation wave is detected in the raw data of the ECG wave as 1, and annotates a point where the fibrillation wave is not detected as 0.

The atrial fibrillation discriminating apparatus 500 may segment morphological characteristics of the fibrillation wave in the ECG wave. The atrial fibrillation discriminating apparatus 500 separately labels and detects a fluctuation of the baseline as a fibrillation wave (tremors).

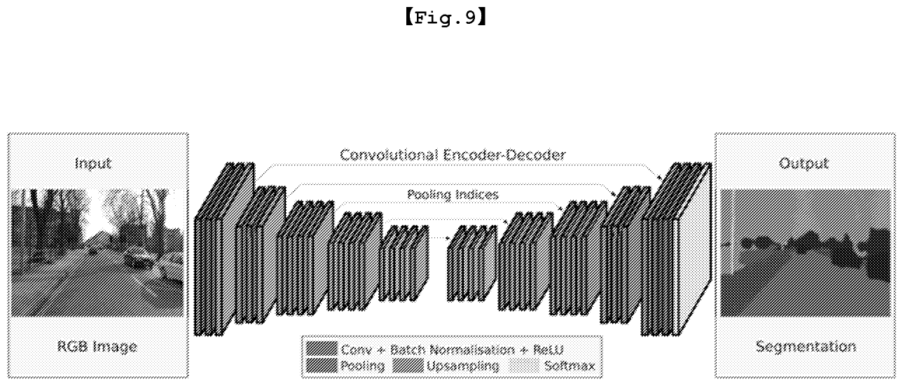

FIG. 9 is a diagram showing a structure of an encoder-decoder segmentation algorithm according to an embodiment of the present invention.

The atrial fibrillation discriminating apparatus 500 generates a segmentation model by performing training using an encoder-decoder type segmentation algorithm. Here, the atrial fibrillation discriminating apparatus 500 generates a segmentation model capable of detecting a section in which a fibrillation wave exists by performing training the encoder-decoder segmentation algorithm. The atrial fibrillation discriminating apparatus 500 continuously accumulates annotated data to update the segmentation model.

The atrial fibrillation discriminating apparatus 500 may label a section corresponding to the fibrillation wave in the ECG wave, and use the result as learning data.

The atrial fibrillation discriminating apparatus 500 designates a certain ROI (Region of Interest) in the ECG wave for actual detection of atrial fibrillation. The atrial fibrillation discriminating apparatus 500 determines what percentage of the fibrillation wave is included in the section corresponding to the ROI. The atrial fibrillation discriminating apparatus 500 determines whether there is atrial fibrillation according to the content of the fibrillation wave in the ROI section. The atrial fibrillation discriminating apparatus 500 determines that the ROI section corresponds to atrial fibrillation in a case where the fibrillation wave is included in the ROI section by a preset threshold (for example, 50%) or greater.

After the labeling is completed, the atrial fibrillation discriminating apparatus 500 moves the ROI section to overlap on the ECG wave. After the labeling is completed, while moving the ROI to overlap on the ECG wave, the atrial fibrillation discriminating apparatus 500 labels, in a case where a window includes the fibrillation wave by the preset threshold (for example, 50%) or greater, the window as atrial fibrillation. The atrial fibrillation discriminating apparatus 500 may detect all sections determined as atrial fibrillation while moving the ROI to overlap on the ECG wave.

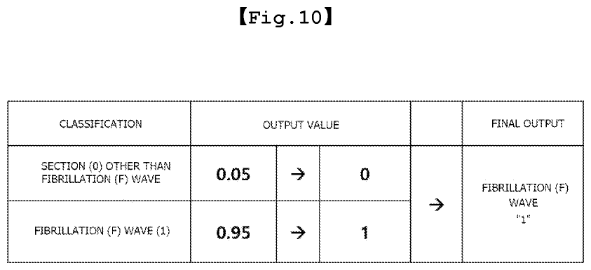

FIG. 10 is a diagram showing a process of outputting a final output value for each point according to an embodiment of the present invention.

The atrial fibrillation discriminating apparatus 500 outputs classification probability values for each class using the segmentation model, and determines, as a final output, a class having the highest classification probability value in each class.

The atrial fibrillation discriminating apparatus 500 annotates, in a case where an output value of a section other than the fibrillation wave is 0.05, the output value as 0. The atrial fibrillation discriminating apparatus 500 annotates, in a case where an output value of a section of the fibrillation wave is 0.95, the output value as 1.

The atrial fibrillation discriminating apparatus 500 determines whether the ratio of the output values annotated as 1 in the whole ECG wave is equal to or greater than a preset ratio (for example, 50%). The atrial fibrillation discriminating apparatus 500 determines that atrial fibrillation has occurred in a case where the ratio of the output value annotated as 1 is equal to or greater than the preset ratio, and labels the final output as 1.

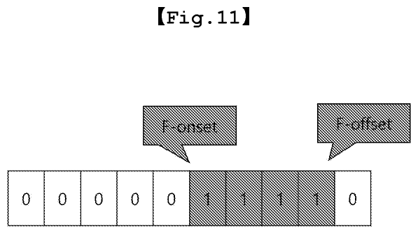

FIG. 11 is a diagram showing a start point and an end point of an F wave according to an embodiment of the present invention.

Since the atrial fibrillation discriminating apparatus 500 outputs a final output value in the point-by-point manner, it is possible to recognize a start point and an end point of the fibrillation wave in the ROI section.

After the labeling is completed while moving the ROI section to overlap on the ECG wave, the atrial fibrillation discriminating apparatus 500 confirms an exact start point (a point first determined as 1) and an exact end point (a point last determined as 1) of the atrial fibrillation.

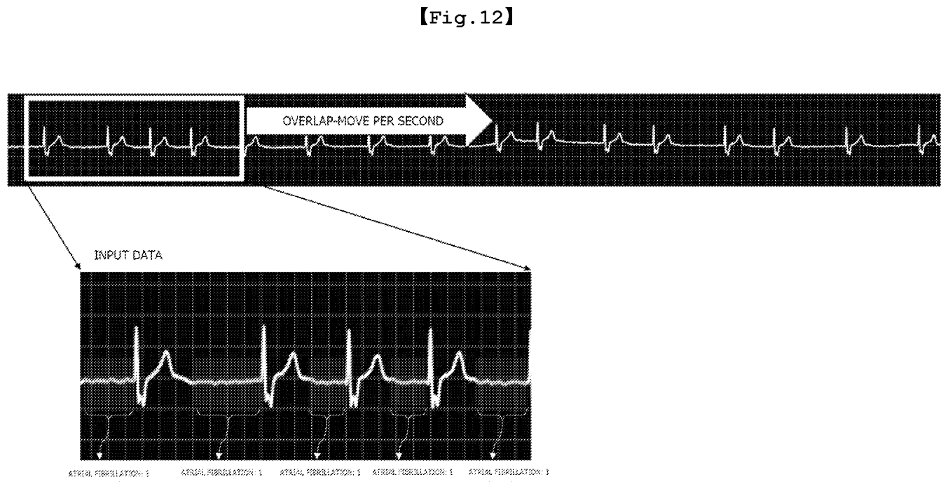

FIG. 12 is a diagram showing point-by-point annotation according to an embodiment of the present invention.

The atrial fibrillation discriminating apparatus 500 calculates an occurrence rate of the fibrillation wave to the size of the entire input data to determine whether atrial fibrillation has occurred in a specific section. The atrial fibrillation discriminating apparatus 500 adjusts a threshold of the occurrence rate value to allow a medical staff to check a probable candidate group and a general candidate group as atrial fibrillation.

The atrial fibrillation discriminating apparatus 500 calculates the occurrence rate of the fibrillation wave section to the size of the input data. For example, in a case where the occurrence rate is 50% or greater, the atrial fibrillation discriminating apparatus 500 classifies a section corresponding to the window as the atrial fibrillation occurrence section. The atrial fibrillation discriminating apparatus 500 may analyze the ECG wave in real time using a segmentation algorithm with a lightweight structure.

The atrial fibrillation discriminating apparatus 500 may move the ROI section on the ECG wave without overlapping on the ECG wave. Preferably, the atrial fibrillation discriminating apparatus 500 detects the fibrillation wave by moving the ROI section to overlap on the ECG wave in order to determine the exact start and end points of the atrial fibrillation.

In the case of early stage patients other than chronic patients, since the fibrillation wave may be detected for only a few seconds (for example, 10 seconds) in measurement for 1 to 3 days, it is preferable to move the ROI section to overlap on the ECG wave to determine the exact start and end points.

In a case where it is determined that the fibrillation wave included in the ROI section is equal to or greater than a preset threshold (for example, 50%), the atrial fibrillation discriminating apparatus 500 labels the ROI section as atrial fibrillation. The atrial fibrillation discriminating apparatus 500 may confirm the start point of atrial fibrillation in a case where the ROI section is determined and labeled as atrial fibrillation. Further, in order to accurately confirm the end point of atrial fibrillation in the ECG wave, the atrial fibrillation discriminating apparatus 500 moves the ROI section per about 1 second to detect the fibrillation wave.

In a case where the atrial fibrillation wave is not detected while moving the ROI section on the ECG wave per about 1 second, the atrial fibrillation discriminating apparatus 500 determines a point at which the atrial fibrillation wave is not detected as the end point of atrial fibrillation.

In a case where the atrial fibrillation wave of the preset threshold (for example, 50%) or greater is detected in the ROI section of the ECG wave and the section is determined as the atrial fibrillation, the atrial fibrillation discriminating apparatus 500 labels the ROI section as 1. Further, in a case where the atrial fibrillation wave below the preset threshold (for example, 50%) is detected in the ROI section of the ECG wave and the section is determined as non-atrial fibrillation, the atrial fibrillation discriminating apparatus 500 labels the ROI section as 0.

The atrial fibrillation discriminating apparatus 500 may determine whether the atrial fibrillation has occurred by checking the proportion of the ROI section determined as the atrial fibrillation and labeled as 1 in the whole ECG wave. The atrial fibrillation discriminating apparatus 500 performs labeling for the atrial fibrillation on the basis of raw data about the ECG wave.

The atrial fibrillation discriminating apparatus 500 performs labeling for atrial fibrillation on the basis of one-dimensional data mapped to indices of raw data. The atrial fibrillation discriminating apparatus 500 checks labeled values mapped to the raw data from the start point to the end point in the ROI section, and checks the ratio of sections with a value of 1 labeled as atrial fibrillation to determine whether there is atrial fibrillation.

The above description is merely an illustrative explanation of the technical idea of the present embodiment, and those skilled in the art will be able to make various modifications and variations without departing from the scope of the invention. Accordingly, the present embodiments are not intended to limit the technical idea of the invention, and the scope of the technical idea of the invention is not limited by these examples. The scope of protection of the invention should be interpreted in accordance with the claims below, and all equivalent technical ideas should be interpreted as being included in the scope of the invention.

1. An atrial fibrillation discriminating apparatus comprising:
an ECG wave acquisition unit that acquires an ECG wave for each of a plurality of a person;
a classification unit that applies segmentation to the ECG wave to check respective sections of the ECG wave, and labels classification values for the sections;
a fibrillation selection unit that selects only a fibrillation wave labeled as tremors on the basis of the classification values for the respective sections; and
an atrial fibrillation determination unit that determines atrial fibrillation in a case where the fibrillation wave of a preset threshold or greater is included.

2. The apparatus according to claim 1, wherein the classification unit checks respective sections of a P wave, a Q wave, an R wave, an S wave, a T wave, and an ROI (Region of Interest) included in the ECG wave.

3. The apparatus according to claim 2, wherein the classification unit labels, in a case where the fibrillation wave of the preset threshold or greater is detected in the ROI section of the ECG wave and the ROI section is determined as the atrial fibrillation, the ROI section as 1, and labels, in a case where the fibrillation wave below the preset threshold is detected in the ROI section and the ROI section is determined as non-atrial fibrillation, the ROI section as 0.

4. The apparatus according to claim 3, wherein the classification unit labels the section having the value of 1 as the atrial fibrillation wave.

5. The apparatus according to claim 1, further comprising a learning unit that performs learning using only the atrial fibrillation wave as input to generate an atrial fibrillation learning result.

6. The apparatus according to claim 2, wherein the classification unit moves the ROI section to overlap on the ECG wave to determine exact start and end points of the atrial fibrillation after the labeling is completed.

7. The apparatus according to claim 6, wherein the classification unit detects the atrial fibrillation wave while moving the ROI section in a preset time unit after the labeling is completed.

8. The apparatus according to claim 6, wherein the classification unit determines a point at which the atrial fibrillation wave is first detected in the ROI section as the start point, and determines a point at which the atrial fibrillation wave is not detected as the end point of atrial fibrillation while moving the ROI section in a preset time unit.

9. The apparatus according to claim 1, wherein the atrial fibrillation determination unit determines whether the atrial fibrillation has occurred by checking the proportion of the ROI section determined as the atrial fibrillation and labeled as 1 in the whole ECG wave.

10. The apparatus according to claim 2, wherein the classification unit detects the atrial fibrillation wave according to an irregularity of an RR interval between the R wave and the next R wave and a morphological shape representing baseline fluctuation, in beat cycles of the ECG wave.
